(19) United States
(12) Patent Application Publication
(10) Pub. No.: US 2009/0053161 A1
(43) Pub. Date: Feb. 26, 2009

(54) PROCESS FOR MANAGING HAIR

(75) Inventors: Nghi Van Nguyen, Edison, NJ (US); Sawa Hashimoto, Westfield, NJ (US); David W. Cannell, Plainfield, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/841,024

(22) Filed: Aug. 20, 2007

(51) Int. Cl. A61K 8/18; A61Q 5/00

(52) U.S. Cl. 424/70.17; 424/70.1

(57) ABSTRACT

The present invention is drawn to a process for managing a keratinous substrate, such as hair, involving applying onto the keratinous substrate an aqueous composition containing at least one compound chosen from a phospholipid, a polyamine, a fatty monoamine, and a fatty quaternary amine; at least one nonionic surfactant; at least one compound chosen from an alkyl(ether)carboxylate having from about 6 to about 40 carbon atoms, and an alkyl(ether)phosphate having from about 6 to about 40 carbon atoms; at least one water-insoluble material; and at least one film former.

### BACKGROUND OF THE INVENTION

The present invention relates to a process for managing hair. The phrase “managing hair” is intended to cover the styling of hair, the promotion of curl retention in hair, and the inhibition of hair fibers, including dyed hair fibers, from becoming frizzy.

### BRIEF SUMMARY OF THE INVENTION

The present invention is drawn to a process for managing a keratinous substrate, such as hair, involving applying onto the keratinous substrate an aqueous composition containing:
(a) at least one compound chosen from a phospholipid, a polyamine, a fatty monoamine, and a fatty quaternary amine;
(b) at least one nonionic surfactant;
(c) at least one compound chosen from an alkyl(ether)carboxylate having from about 6 to about 40 carbon atoms, and an alkyl(ether)phosphate having from about 6 to about 40 carbon atoms;
(d) at least one water-insoluble material; and
(e) at least one film former.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

The term “water-insoluble” means those compounds which are either completely or partially insoluble in water.

The term “carried” means that the aqueous delivery system containing the water-insoluble ingredients is both homogeneous and clear to substantially clear in appearance.

“At least one” as used herein means one or more and thus includes individual components as well as mixtures/combinations.

“Formed from,” as used herein, means obtained from chemical reaction of, wherein “chemical reaction,” includes spontaneous chemical reactions and induced chemical reactions. As used herein, the phrase “formed from”, is open ended and does not limit the components of the composition to those listed, e.g., as component (i) and component (ii). Furthermore, the phrase “formed from” does not limit the order of adding components to the composition or require that the listed components (e.g., components (i) and (ii)) be added to the composition before any other components.

“Hydrocarbons,” as used herein, include alkanes, alkenes, and alkynes, wherein the alkanes comprise at least one carbon, and the alkenes and alkynes each comprise at least two carbons; further wherein the hydrocarbons may be chosen from linear hydrocarbons, branched hydrocarbons, and cyclic hydrocarbons; further wherein the hydrocarbons may optionally be substituted; and further wherein the hydrocarbons may optionally further comprise at least one heteroatom intercalated in the hydrocarbon chain.

“Silicone compound,” as used herein, includes, for example, silica, silanes, silazanes, siloxanes, and organosiloxanes; and refers to a compound comprising at least one silicon; wherein the silicone compound may be chosen from linear silicone compounds, branched silicone compounds, and cyclic silicone compounds; further wherein the silicone compound may optionally be substituted; and further wherein the silicone compound may optionally further comprise at least one heteroatom intercalated in the silicone chain, wherein the at least one heteroatom is different from the at least one silicon.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups.

The substituent(s) may be further substituted.

“Ethylene oxide group” as defined herein refers to a group of formula —CH₂CH₂—O—.

“Propylene oxide group” as defined herein includes groups of formula —CH₂CH₂CH₂—O—, groups of formula (CH₃)CHCH₂—O—, and groups of formula —CH₂(CH₃)CH—O—.

“Keratinous substrate” as defined herein may be human keratinous fiber, and may be chosen from, for example, hair, eyelashes, and eyebrows, as well as the stratum corneum of the skin and nails.

“Polymers,” as defined herein, include homopolymers and copolymers formed from at least two different types of monomers.

Advantageously, the aqueous composition of the present invention enables water-insoluble materials and film forming agents to be carried by the composition and yet provide a clear to substantially clear appearance. Surprisingly, the use of an alcohol is not required in order to render the composition clear to substantially clear in appearance.

Phospholipid

The present invention provides for the use of conventional organic phospholipids. Particularly preferred organic phospholipids include lecithins. Lecithins are mixtures of phospholipids, i.e., diglycerides of fatty acids linked to an ester of phosphoric acid. Preferably, lecithins are diglycerides of stearic, palmitic, and oleic acids linked to the choline ester of phosphoric acid. Lecithin is usually defined either as pure phosphatidyl cholines or as crude mixtures of phospholipids which include phosphatidyl choline, phosphatidyl serine, phosphatidyl ethanolamine, phosphatidyl inositol, other phospholipids, and a variety of other compounds such as fatty acids, triglycerides, sterols, carbohydrates, and glycolipids.

The lecithin used in the present invention may be present in the form of a liquid, powder, or granules. Lecithins useful in the invention include, but are not limited to, soy lecithin and hydroxylated lecithin. For example, ALCOLEC S is a fluid soy lecithin, ALCOLEC F 100 is a powder soy lecithin, and ALCOLEC Z3 is a hydroxylated lecithin, all of which are available from the American Lecithin Company.

Other than lecithins, additional examples of phospholipids which may be useful in the present invention include, but are not limited to, multifunctional biomimetic phospholipids. For example, the following multifunctional biomimetic phospholipids manufactured by Uniqema Industries may be useful: PHOSPHOLIPID PTC, PHOSPHOLIPID CDM, PHOSPHOLIPID SV, PHOSPHOLIPID GLA, and PHOSPHOLIPID EFA.

In the present invention, the at least one phospholipid compound may be used in an amount of from greater than 0% to about 30% by weight, preferably from greater than 0% to about 10% by weight, and more preferably from greater than 0% to about 5% by weight, based on the weight of the composition as a whole.

Polyamine

The at least one polyamine compound of the present invention comprises at least three amino groups; preferably at least 4 amino groups; preferably at least 5 amino groups; preferably at least 10 amino groups.

In one embodiment of the present invention, the at least one polyamine compound may, for example, be chosen from aminated polysaccharides comprising at least three amino groups, such as, for example, hydrolysates of aminated polysaccharides comprising at least three amino groups. In one embodiment, the at least one polyamine compound may, for example, be chosen from polymers. Suitable polymers for use as the at least one amine compound are polymers comprising at least three amino groups as defined herein. Non-limiting examples of suitable polymers include homopolymers comprising at least three amino groups, copolymers comprising at least three amino groups, and terpolymers comprising at least three amino groups. Thus, the at least one polyamine compound comprising at least three amino groups may be chosen from, for example, polymers comprising at least three amino groups formed from (i) at least one monomer unit comprising at least one amino group as defined herein, and, optionally, (ii) at least one additional monomer unit different from the at least one monomer (i) ; and polymers comprising at least three amino groups formed from (i) at least one monomer comprising at least three amino groups as defined herein, and, optionally, (ii) at least one additional monomer unit different from the at least one monomer (i). According to the present invention, the at least one additional monomer different from the at least one monomer (i) may or may not comprise at least one amino group as defined herein.

In one embodiment, polyamines are chosen from polyethyleneimines (also commonly designated as PEI). Polyethyleneimines suitable for use in the compositions of the present invention may optionally be substituted. Non-limiting examples of polyethyleneimines which may be used in the composition according to the present invention are the Lupasol® products commercially available from BASF. Suitable examples of Lupasol® polyethyleneimines include Lupasol® PS, Lupasol® PL, Lupasol® PR8515, Lupasol® G20, Lupasol® G35 as well as Lupasol® SC Polythyleneimine Reaction Products (such as Lupasol® SC-61B, Lupasol® SC-62J, and Lupasol® SC-86X). Other non-limiting examples of polyethyleneimines which may be used in the composition according to the present invention are the Epomin® products commercially available from Aceto. Suitable examples of Epomin® polyethyleneimines include Epomin® SP-006, Epomin® SP-012, Epomin® SP-018, and Epomin® P-1000.

Polyamines suitable for use in the present invention may also be chosen from polyvinylamines. Examples thereof include Lupamine 9095, 9030, 9010, 5095, 1595 from BASF.

Another suitable polyamine is an amine substituted polyacrylate crosspolymer includes Carbopol Aqua CC polymer from Noveon, Inc.

The polyamine compounds can also be substituted. An example of such a compound is PEG-15 Cocopolyamine from Cognis.

In another embodiment, the at least one polyamine compound comprising at least three amino groups is chosen from proteins and protein derivatives. Non-limiting examples of suitable proteins and protein derivatives for use in the present invention include those listed at pages 2719 to 2722 of the C.T.F.A. International Cosmetic Ingredient Dictionary and Handbook, 11^{th} edition, vol. 3, (2006). In one embodiment, the at least one polyamine compound comprising at least three amino groups is chosen from wheat protein, soy protein, oat protein, collagen, and keratin protein.

In one embodiment, the at least one polyamine compound comprising at least three amino groups is not chosen from proteins and protein derivatives. In one embodiment, the at least one polyamine compound comprising at least three amino groups is not chosen from compounds comprising lysine, compounds comprising arginine, and compounds comprising histidine. In one embodiment, the at least one polyamine compound comprising at least three amino groups is chosen from compounds comprising lysine, compounds comprising arginine, compounds comprising histidine, and compounds comprising hydroxylysine.

In the present invention, the at least one polyamine compound may be used in an amount of from greater than 0% to about 30% by weight, preferably from greater than 0% to about 10% by weight, and more preferably from greater than 0% to about 5% by weight, based on the weight of the composition as a whole.

Fatty Monamine

The present invention provides for the use of conventional fatty monoamine compounds. Fatty monoamine compounds are those which have more than one hydrocarbon group with from 6 to 22 carbon atoms. Primary, secondary, and tertiary fatty monoamines are useful. Particularly useful are tertiary amido amines having an alkyl group of from about 6 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethyl amine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachnidamidopropyldimethylamine, arachidamidopropyldiethylamine, arachidamidoethyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, hydroxylated, ethoxylated or propoxylated fatty amines such as ethoxylated stearylamine, dihydroxyethylstearylamine, and arachidylbehenylamine. Useful amines in the present invention are disclosed in U.S. Pat. No. 4,275,055.

In the present invention, the at least one fatty monoamine compound may be used in an amount of from greater than 0% to about 30% by weight, preferably from greater than 0% to about 10% by weight, and more preferably from greater than 0% to about 5% by weight, based on the weight of the composition as a whole.

Fatty Quaternary Amines

The present invention provides for the use of conventional fatty quaternary amine compounds containing from about 6 to about 22 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, benzyl or short chain alkyl or hydroxyalkyl groups such as methyl, ethyl, hydroxymethyl or hydroxyethyl, hydroxypropyl or combinations thereof.

Examples of fatty quaternary ammonium compounds include but are not limited to: Behentrimonium chloride, Cocotrimonium chloride, Cethethyldimonium bromide, Dibehenyldimonium chloride, Dihydrogenated tallow benzylmonium chloride, disoyadimonium chloride, Ditallowdimonium chloride, Hydroxycetyl hydroxyethyl dimonium chloride, Hydroxyethyl Behenamidopropyl dimonium chloride, Hydroxyethyl Cetyldimonium chloride, Hydroxyethyl tallowdimonium chloride, myristalkonium chloride, PEG-2 oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryldimonium chloride, cetalkonium chloride, dicetyldimonium chloride, soyatrimonium chloride, stearyl octyl dimonium methosulfate, behentrimonium methosulfate (18-MEA), stearalkonium chloride, and mixtures thereof. Other quaternary ammonium compounds are listed in the CTFA Cosmetic Ingredient Handbook, 11th Edition, on pages 2723-2726, incorporated herein by reference.

In the present invention, the at least one fatty quaternary amine compound may be used in an amount of from greater than 0% to about 30% by weight, preferably from greater than 0% to about 10% by weight, and more preferably from greater than 0% to about 5% by weight, based on the weight of the composition as a whole.

Nonionic Surfactant

In general, nonionic surfactants having a Hydrophilic-Lipophilic Balance (HLB) of from 8 to 20 are contemplated for use by the present invention. Nonlimiting examples of nonionic surfactants useful in the compositions of the present invention are disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C₁₂-C₅₀ range, preferably in the C₁₆-C₄₀ range, more preferably in the C₂₄ to C₄₀ range, and having from about 1 to about 110 alkoxy groups. The alkoxy groups are selected from the group consisting of C₂-C₆ oxides and their mixtures, with ethylene oxide, propylene oxide, and their mixtures being the preferred alkoxides. The alkyl chain may be linear, branched, saturated, or unsaturated. Of these alkoxylated non-ionic surfactants, the alkoxylated alcohols are preferred, and the ethoxylated alcohols and propoxylated alcohols are more preferred. The alkoxylated alcohols may be used alone or in mixtures thereof. The alkoxylated alcohols may also be used in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij nonionic surfactants from Uniqema, Wilmington, Del. Typically, Brij is the condensation products of aliphatic alcohols with from about 1 to about 54 moles of ethylene oxide, the alkyl chain of the alcohol being typically a linear chain and having from about 8 to about 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, e.g. C₈-C₃₀ alcohols, with sugar or starch polymers. These compounds can be represented by the formula (S)ₙ—O—R wherein S is a sugar moiety such as glucose, fructose, mannose, galactose, and the like; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APG 325 CS) and lauryl polyglucoside (available as APG 600CS and 625 CS) , all the above-identified polyglucosides APG are available from Cognis, Ambler, Pa. Also useful herein are sucrose ester surfactants such as sucrose cocoate and sucrose laurate.

Other nonionic surfactants suitable for use in the present invention are glyceryl esters and polyglyceryl esters, including but not limited to, glyceryl monoesters, preferably glyceryl monoesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C₁₆-C₂₂ saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Uniqema), sorbitan monoisostearate (e.g., CRILL® 6 from Croda, Inc., Edison, N.J.), sorbitan stearates (e.g., SPAN® 60), sorbitan trioleate (e.g., SPAN 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use herein are alkoxylated derivatives of glyceryl esters, sorbitan esters, and alkyl polyglycosides, wherein the alkoxy groups is selected from the group consisting of C₂-C₆ oxides and their mixtures, with ethoxylated or propoxylated derivatives of these materials being the preferred. Nonlimiting examples of commercially available ethoxylated materials include TWEEN® (ethoxylated sorbitan mono-, di- and/or tri-esters of C₁₂ to C₁₈ fatty acids with an average degree of ethoxylation of from about 2 to about 20).

Preferred nonionic surfactants are those formed from a fatty alcohol, a fatty acid, or a glyceride with a C₄ to C₃₆ carbon chain, preferably a C₁₂ to C₁₈ carbon chain, more preferably a C₁₆ to C₁₈ carbon chain, derivatized to yield an HLB of at least 8. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 8. Preferably the nonionic surfactants contain ethoxylate in a molar content of from about 10-25, more preferably from about 10-20 moles.

The nonionic surfactant will typically be present in the composition in an amount of from greater than 0% to about 70% by weight, preferably from greater than 0% to 40% by weight, and more preferably from greater than 0% to 20% by weight, based on the weight of the composition as a whole.

Alykyl(ether)carboxylate

The alkyl(ether)carboxylic acid or alkyl(ether)carboxylate used in the present invention corresponds to formula I:

RO[CH₂O]ᵤ[(CH₂)ₓCH(R′)(CH₂)_{y}(CH₂)_{z}O]ᵥ[CH₂CH₂O]_{w}CH₂COOM (I)

wherein:
R is a hydrocarbon radical containing from 6 to 40 carbon atoms;
u, v and w, independently of one another, represent numbers of from 0 to 60;
x, y and z, independently of one another, represent numbers of from 0 to 13;
R′ represents hydrogen, alkyl, the sum of x+y+z being ┌ . . . 0;
M is an alkali metal or alkaline earth metal (i.e., ether carboxylate) or hydrogen (i.e., ether carboxylic acid).

Ether carboxylic acids or carboxylates corresponding to formula (I) can be obtained by alkoxylation of alcohols ROH with ethylene oxide as sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (I), R is linear or branched, acyclic or cyclic, saturated or unsaturated, aliphatic or aromatic, substituted or unsubstituted, preferably a linear or branched, acyclic C₆₋₄₀ alkyl or alkenyl group or a C₁₋₄₀ alkyl phenyl group, more particularly a C₈-C₂₂ alkyl or alkenyl group or a C₄-C₁₈ alkyl phenyl group, more preferably a C₁₂-C₁₈ alkyl group or alkenyl group or a C₆-C₁₆ alkyl phenyl group; u, v, w, independently of one another, is preferably a number from 2 to 20, more preferably a number from 3 to 17 and most preferably a number from 5 to 15;

x, y, z, independently of one another, is preferably a number from 2 to 13, more preferably a number from 1 to 10 and most preferably a number from 0 to 8; M may be chosen from lithium, sodium, potassium, calcium, magnesium or hydrogen.

Suitable ether carboxylic acids or ether carboxylates include, but are not limited to, the following representatives referred to by their INCI names (INCI: nomenclature for raw materials according to the International Cosmetic Ingredient Dictionary, 11th Edition, published by the Cosmetic, Toiletry and Fragrance Association Inc. (CTFA), Washington D.C., USA): Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C₁₁-C₁₅ Pareth-7 Carboxylic Acid, C₁₂-C₁₃ Pareth-5 Carboxylic Acid, C₁₂-C₁₃ Pareth-8 Carboxylic Acid, C12-C13 Pareth-12 Carboxylic Acid, C₁₂-C₁₅ Pareth-7 Carboxylic Acid, C₁₂-C₁₅ Pareth-8 Carboxylic Acid, C₁₄-C₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, Magnesium Laureth-11 Carboxylate, Sodium-PPG-6-Laureth-6-Carboxylate, Sodium PPG-8-Steareth-7 Carboxylate, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Sodium Capryleth-2 Carboxylate, Sodium Capryleth-9 Carboxylate, Sodium Ceteth-13 Carboxylate, Sodium C9-11 Pareth-6 Carboxylate, Sodium C11-C15 Pareth-7 Carboxylate, Sodium C12-C13 Pareth-5 Carboxylate, Sodium C12-C13 Pareth-8 Carboxylate, Sodium C12-C13 Pareth-12 Carboxylate, Sodium C12-C15 Pareth-6 Carboxylate, Sodium C12-C15 Pareth-7 Carboxylate, Sodium C12-C15 Pareth-8 Carboxylate, Sodium C14-C15 Pareth-8 Carboxylate, Sodium Deceth-2 Carboxylate, Sodium Hexeth-4 Carboxylate, Sodium Isosteareth-6 Carboxylate, Sodium Isosteareth-11 Carboxylate, Sodium Laureth-3 Carboxylate, Sodium Laureth-4 Carboxylate, Sodium Laureth-5 Carboxylate, Sodium Laureth-6 Carboxylate, Sodium Laureth-8 Carboxylate Sodium Laureth-11 Carboxylate, Sodium Laureth-12 Carboxylate, Sodium Laureth-13 Carboxylate, Sodium Laureth-14 Carboxylate, Sodium Laureth-17 Carboxylate, Sodium Trudeceth-3 Carboxylate, Sodium Trideceth-6 Carboxylate, Sodium Trideceth-7 Carboxylate, Sodium Trideceth-8 Carboxylate, Sodium Trideceth-12 Carboxylate, Sodium Undeceth-5 Carboxylate, Trideceth-3 Carboxylic Acid, Trideceth4 Carboxylic Acid, Trideceth-7 Carboxylic acid, Trideceth-15 Carboxylic Acid, Trideceth-19 Carboxylic Acid, Undeceth-5 Carboxylic Acid.

Particularly preferred are oleth-10 carboxylic acid, laureth-5 carboxylic acid, and laureth-11 carboxylic acid.

In the present invention, the alkyl(ether)carboxylic acids or alkyl(ether)carboxlyates may be used in an amount of from greater than 0% to about 30% by weight, preferably from greater than 0% to about 10% by weight, and more preferably from greater than 0% to about 5% by weight, based on the weight of the composition as a whole.

The alkyl(ether)carboxylate used in the present invention include alkyl carboxylates and alkyl carboxylic acids.

Non-limiting examples of alkyl carboxylates or alkyl carboxylic acids includes fatty acids having from about 6 to about 40 carbon atoms corresponding to formula (II)

RCOOM (II)

wherein:

R is a hydrocarbon radical containing from about 6 to about 40 carbon atoms. In addition, R is linear or branched, acyclic or cyclic, saturated or unsaturated, aliphatic or aromatic, substituted or unsubstituted. Typically, R is a linear or branched, acyclic C₆₋₄₀ alkyl or alkenyl group or a C₁₋₄₀ alkyl phenyl group, more typically a C₈₋₂₂ alkyl or alkenyl group or a C₄₋₁₈ alkyl phenyl group, and even more typically a C₁₂₋₁₈ alkyl group or alkenyl group or a C₆₋₁₆ alkyl phenyl group; and M is an alkali metal or alkaline earth metal (i.e., carboxylate) or hydrogen (i.e., carboxylic acid).

Suitable fatty acids having from about 6 to about 40 carbon atoms include, but are not limited to the following representatives referred to by their INCI names (INCI: nomenclature for raw materials according to the International Cosmetic Ingredient Dictionary, 11^{th} Edition, published by the Cosmetic, Toiletry and Fragrance Association Inc. (CTFA), Washington D.C., USA): Arachidic Acid, Arachidonic Acid, Beeswax Acid, Capric Acid, Caproic Acid, Caprylic Acid, Coconut Acid, Isostearic Acid, Lauric Acid, Linoleic Acid, Linolenic Acid, Myristic Acid, Oleic Acid, Olive Acid, Palmitic Acid, Rapeseed Acid, Stearic Acid, Tallow Acid, Undecanoic Acid, Undecylenic Acid or Wheat Germ Acid and mixtures thereof.

Typical fatty acids having from about 6 to about 40 carbon atoms include Capric Acid, Caprylic Acid, Lauric Acid, Oleic Acid, Isostearic Acid, and Stearic Acid.

In the present invention, the alkyl carboxylic acids or carboxlyates may be used in an amount of from greater than 0% to about 30% by weight, preferably from greater than 0% to about 10% by weight, and more preferably from greater than 0% to about 5% by weight, based on the weight of the composition as a whole.

Alkyl(ether)phosphates

Suitable alkyl(ether)phosphates include, but are not limited to, alkoxylated alkyl phosphate esters and alkyl phosphate esters corresponding to a mono-ester of formula (III) and salts thereof:

RO[CH₂O]ᵤ[(CH₂)ₓCH(R′)(CH₂)_{y}(CH₂)_{z}O]ᵥ[CH₂CH₂O]_{w}—PO—(OH)₂ Formula (III);

a di-ester corresponding to formula (IV) and salts thereof:

{RO[CH₂O]ᵤ[(CH₂)ₓCH(R′)(CH₂)_{y}(CH₂)_{z}O]ᵥ[CH₂CH₂O]_{w}}₂PO—(OH) Formula (IV);

a tri-ester corresponding to formula (V):

{RO[CH₂O]ᵤ[(CH₂)ₓCH(R′)(CH₂)_{y}(CH₂)_{z}O]ᵥ[CH₂CH₂O]_{w}}₃PO Formula (V);

and combinations thereof,

wherein:
R is a hydrocarbon radical containing from 6 to 40 carbon atoms;
u, v and w, independently of one another, represent numbers of from 0 to 60;
x, y and z, independently of one another, represent numbers of from 0 to 13;
R′ represents hydrogen, alkyl, the sum of x+y+z being ≧0.
   The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formulas (III), (IV) and (V) , R is linear or branched, acyclic or cyclic, saturated or unsaturated, aliphatic or aromatic, substituted or unsubstituted, preferably a linear or branched, acyclic C₆₋₄₀ alkyl or alkenyl group or a C₁₋₄₀ alkyl phenyl group, more particularly a C₈₋₂₂ alkyl or alkenyl group or a C₄₋₁₈ alkyl phenyl group, more preferably a C₁₂₋₁₈ alkyl group or alkenyl group or a C₆₋₁₆ alkyl phenyl group; u, v, w, independently of one another, is preferably a number from 2 to 20, more preferably a number from 3 to 17 and most preferably a number from 5 to 15;

x, y, z, independently of one another, is preferably a number from 2 to 13, more preferably a number from 1 to 10 and most preferably a number from 0 to 8.

In general, the lower the number of carbon atoms in the R group of the phosphate esters, the more irritating to the skin and the less soluble in water the phosphate ester becomes. In contrast, the higher the number of carbon atoms in the R group, the milder to the skin and the thicker and more waxy the resultant product becomes. Accordingly, for best results, R should have from 12 to 18 carbon atoms.

Particularly preferred alkoxylated alkyl phosphate esters for use in the present invention are PPG-5-Ceteth-10 phosphate (CRODAFOS SG®), Oleth-3 phosphate (CRODAFOS N3 acid), Oleth-10 phosphate (CRODAFOS N10 acid), and a mixture of Ceteth-10 phosphate and Dicetyl phosphate (CRODAFOS CES) all sold by Croda. Particularly preferred alkyl phosphate esters are Cetyl phosphate (Hostaphat CC 100), Stearyl phosphate (Hostaphat CS 120) from Clariant.

In the present invention, the alkyl(ether)phosphates may be used in an amount of from greater than 0% to about 30% by weight, preferably from greater than 0% to about 10% by weight, and more preferably from greater than 0% to about 5% by weight, based on the weight of the composition as a whole.

Water-Insoluble Materials

Water-insoluble materials or ingredients include, but are not limited to, the following:

Lipophilic “ingredients” or “materials” such as silicones, oil-soluble vitamins such as Vitamin E and Vitamin A, sunscreens, ceramides and natural oils: The lipophilic ingredients may be in the form of sunscreens, bacteriostats, moisturizers, colors, topical pharmaceuticals and the like. Preferred lipophilic ingredients include: Vitamin E, Vitamin E Acetate, Vitamin A Palmitate, olive oil, mineral oil, 2-oleamido-1,3-octadecanediol, octylmethoxy cinnamate, octyl salicylate, and silicones such as dimethicone, cyclomethicone, phenyl trimethicone, dimethiconol, dimethicone copolyol, aminosilicone and laurylmethicone copolyol. The lipophilic ingredients will, for example, moisturize or condition the skin, hair, and/or eyelashes and leave behind no oily feel.

Water-insoluble polymers, resins, and latexes, wherein the polymers and resins include but are not limited to those containing carboxyl moieties, such as acrylates and other carboxy polymers.

Preferred water-insoluble ingredients for use in the present invention include silicones ranging from low molecular weight fluids to high molecular weight gums; hydrocarbons such as mineral oil, petrolatum, paraffins, iso-paraffins, aromatic hydrocarbons, and the like; plant oils such as olive, avocado, coconut, and the like; fatty acids; fatty esters; fatty alcohols; and fatty waxes.

In the present invention, the water-insoluble materials are used in an amount of from greater than 0% to about 50% by weight, preferably from greater than 0% to about 20% by weight, and more preferably from greater than 0% to about 10% by weight, based on the weight of the composition as a whole

Film Formers

The composition of the present invention may also contain at least one film-forming polymer in order to impart styling and curl retention properties onto the hair. Film-forming polymers useful herein are neutralized, non-neutralized or partially neutralized, polymers and resins, wherein the polymers and resins include but are not limited to those containing carboxyl moieties, such as acrylates and other carboxy polymers. Examples of suitable water soluble film forming polymers include, for example, PVP, PVP/VA, acrylates, polyesters, polyurethranes, polyimides, polysulfonates, guars, starches and the like. Typically, water-insoluble polymers and resins have to be neutralized to about 90% of their carboxyl moieties to make them water soluble for the purpose of formulating products in aqueous solution and for the purpose of making products which have good non-build-up properties, i.e., can be easily washed off the hair after use.

The following are examples of film forming polymers that can be employed by the present invention. The list is not intended to be limiting:
AMPHOMER LV-71 from National Starch (octylacrylamide/acrylates/butylaminoethyl methacrylate copolymer),
OMNIREZ-2000 from ISP (PVM/MA half ethyl ester copolymer),
RESYN 28-2930 from National Starch (Vinyl acetate/crotonates/vinyl neodecanoate copolymer),
LUVIMER 100P from BASF (t-butyl acrylate/ethyl acrylate/methacrylic acid), and
ULTRAHOLD STRONG from BASF (acrylic acid/ethyl acrylate/t-butyl acrylamide),
SALCARE SC60 from Ciba (Acrylamidopropyltrimonium Chloride/Acrylamide Copolymer),
BALANCE CR from National Starch (Acrylates Copolymer),
AMPHOMER 28-4961 from National Starch (Acrylates/Octylacrylamide Copolymer),
TORAY SETSIL 301 from Dow Corning (Acrylates/Octylacrylamide/Diphenyl Amodimethicone Copolymer),
DIAFORMER Z-632N from Clariant (Acrylates/Stearyl Acrylate/Ethylamine Oxide Methacrylate Copolymer),
ULTRAHOLD 8 from BASF (Acrylates/t-Butylacrylamide Copolymer),
MEXOMERE PQ from Chimex (Allyl Stearate/VA Copolymer),
FIXATE G-100 from Noveon (AMP-Acrylates/Allyl Methacrylate Copolymer),
GANTREZ A-425 from ISP (Butyl Ester of PVM/MA Copolymer),
GANEX P-904 from ISP (Butylated PVP),
AMAZE from National Starch (Corn Starch Modofied),
MEXOMERE PL from Chimex (Diethylene Glycolamine/Epichlorohydrin/Piperazine Copolymer),
EASTMAN AQ POLYMER from Eastman (Diglycol/CHDM/Isophthalate/SIP Copolymer),
JAGUAR C 13S from Rhodia (Guar Hydroxylpropyl Trimonium Chloride),
AQUAFLEX FX-64 from ISP (Isobutylene/Ethylmaleimide/Hydroxyethylmaleimide Copolymer),
LUVIFLEX SILK from BASF (PEG/PPG-25/25 Dimethicone/Acrylates Copolymer),
AQUAFLEX XL-30 from ISP (Polyimide-1),
LUVISET P.U.R from BASF (Polyurethrane-1),
LUVISKOL PLUS from BASF (Polyvinylcaprolactam),
AQUAFLEX SF-40 from ISP (PVP/Vinylcaprolactam/DMAPA Acrylates Copolymers),
ADVANTAGE PLUS from ISP (VA/Butyl Maleate/Isobornyl Acrylate Copolymer),
MEXOMERE PW from Chimex (VA/Vinyl Butyl Benzoate/Crotonates Copolymer),
GAFFIX VC-713 from ISP (Vinyl Caprolactam/VP/Dimethylaminoethyl Methacrylate Copolymer),
COPOLYMER 845 from ISP (VP/Dimethylaminoethylmethacrylate Copolymer),
GANEX V-516 from ISP (VP/Hexadecene Copolymer), LUVISKOL VA 64 from BASF (VP/VA Copolymer).
   Unneutralized or partially neutralized water-insoluble latexes can also be used as invention film-forming polymers. Included are the following latexes:
AMERHOLD DR-25 from Amerchol (acrylic acid/methacrylic acid/acrylates/methacrylates),
LUVIMER 36D from BASF (ethyl acrylate/t-butyl acrylate/methacrylic acid), and
ACUDYNE 258 from Rohm & Haas (acrylic acid/methacrylic acid/acrylates/methacrylates/hydroxy ester acrylates).

The film forming polymer may be employed in an amount sufficient to impart and/or maintain a shape on the hair. Typically, it will be employed in an amount of from greater than 0 to 30% by weight, preferably from 1 to 10% by weight, and more preferably from 1 to 5% by weight, based on total weight of composition.

The composition can contain additional ingredients such as anionic surfactants, organic salts, inorganic salts, proteins, hair dyes, water-soluble polymers, quaternary ammonium compounds, complex and simple carbohydrates, amino acids, preservatives and fragrances.

The composition of the present invention may be used to formulate products for styling hair, curl retention and anti-frizz.

The invention will be further clarified by the following examples, which are intended to be illustrative of the invention, but not limiting thereof.

### EXAMPLES

### Example 1

### Anti-Frizz Properties of the Disclosed Compositions Below as Rinse-Off Treatments

Hair swatches (commercial normal brown hair, 8 inches long, 0.35 g hair/swatch) were massaged with the following solutions (0.5 g product/swatch) for 15 seconds and allowed to stand at room temperature for 1 minute:
A. PVP K90 (3%)
B. PEI (5%), Procetyl AWS¹(20%), Laureth-5 Carboxylic Acid (2%), Mineral Oil (3%)
C. PEI (5%), Procetyl AWS (20%), Laureth-5 Carboxylic Acid (2%), Mineral Oil (3%), PVP K90 (3%)
D. Amphomer LV-71², neutralized 100% with AMP (6%)
E. Lecithin (5%), Procetyl AWS (25%), Laureth-11 Carboxylic Acid (1%), Olive Oil (2%)
F. Lecithin (5%), Procetyl AWS (25%), Laureth-11 Carboxylic Acid (1%), Olive Oil (2%), Amphomer LV-71, neutralized 100% with AMP (6%)
G. Resyn 2829-303, neutralized 100% with AMP
H. Behentrimonium Chloride (1%), Procetyl AWS (25%), Crodafos NlOAcid (1%), Mineral Oil (2%)
I. Behentrimonium Chloride (1%), Procetyl AWS (25%), Crodafos NlOAcid (1%), Mineral Oil (2%), Resyn 2829-30, neutralized 100% with AMP
   All solutions were adjusted to 100% with water. ¹Procetyl AWS is PPG-5-Ceteth-20²Amphomer LV-71 is Octylacrylamide/acrylates/butylaminoethyl methacrylate copolymer³Resyn 2829-30 is Vinyl acetate/crotonates/vinyl neodecanoate copolymer

The treated hair swatches were rinsed with water for 10 seconds, wounded around the pegboards, then placed in the oven at 50° C. for 1 hour. After equilibrating at room temperature overnight, the hair swatches were removed from the pegboard (t0) and placed in the humidity chamber (95% RH) for 4 hours (t4). The change in the total area of the swatches (determined by an image analyzer) from t0 to t4 represents the frizz of the hair swatches. The results are shown in Table 1.

TABLE 1
Anti-Frizz Measurements using the Disclosed
Compositions

| Treatment | % Change in Area |
|---|---|
| A | 1399.29 |
| B | 389.52 |
| C | 101.92 |
| D | 1531.77 |
| E | 207.89 |
| F | 139.77 |
| G | 1479.87 |
| H | 237.51 |
| I | 134.88 |

A higher percent change in the total area represents greater frizz for the hair under high humidity. The data indicate that the disclosed inventive compositions containing film formers (C, F, and I) showed significantly less percent changes in the area of the hair swatches indicating a significant improvement in the anti-frizz effect of the inventive compositions compared to those disclosed compositions that contained the film former alone or that did not contain the film formers.

### Example 2

### Durability of the Anti-Frizz Properties of the Disclosed Compositions

The hair swatches from Example 1 were rinsed with water for 10 seconds. The rinsed hair swatches were then wound on the pegboard and the percent change in the total area of the hair swatches were calculated as described above. The results are depicted in Table 2.

TABLE 2
Durability of the Anti-Frizz Properties of
the Disclosed Compositions

| Treatment | % Change in Area |
|---|---|
| A | 1063.53 |
| B | 832.31 |
| C | 229.02 |
| D | 1078.24 |
| E | 677.75 |
| F | 375.92 |
| G | 1088.62 |
| H | 772.13 |
| I | 320.37 |

The data indicate that even after rinsing, the hair treated with the disclosed inventive compositions (C, F, and I) showed significantly better anti-frizz properties than the hair swatches treated with the film former alone or with the disclosed compositions without the film former.

### Example 3

### Anti-Frizz Properties of the Disclosed Composition as a Shampoo

Following the procedure described above, hair swatches were treated (0.5 g shampoo/swatch, massage in for 15 seconds, wait for 1 minute, then rinse with water for 10 seconds) with the following shampoos:

Shampoo A: SLES (12%), Lexamine S-13 (stearamidopropyldimethylamine) (2%), Procetyl AWS (3%), Crodafos N3A (1%), Olive Oil (1%) , Polymer JR 30M (polyquaternium-10)(1.5%), qs to 100% with water.

Shampoo B: SLES (12%), Lexamine S-13 (stearamidopropyldimethylamine) (2%), Procetyl AWS (3%), Crodafos N3A (1%), Olive Oil (1%), Polymer JR 30M (polyquaternium-10)(1.5%), Amphomer LV-71 (3%), qs to 100% with water

The results are shown in Table 3.

TABLE 3
Anti-Frizz of the Disclosed Compositions as
a Shampoo

| Shampoo | % Change in Area |
|---|---|
| A | 670.75 |
| B | 187.35 |

The results show that hair shampooed with the disclosed inventive composition (B) exhibited significantly better anti-frizz property than the one shampooed with the disclosed composition without the film former (A).

1. A process for managing a keratinous substrate, such as hair, involving applying onto the keratinous substrate an aqueous composition comprising:
(a) at least one compound chosen from a phospholipid, a polyamine, a fatty monoamine, and a fatty quaternary amine;
(b) at least one nonionic surfactant;
(c) at least one compound chosen from an alkyl(ether)carboxylate having from about 6 to about 40 carbon atoms, and an alkyl(ether)phosphate having from about 6 to about 40 carbon atoms;
(d) at least one water-insoluble material; and
(e) at least one film former.

2. The process of claim 1 wherein (a) is a phospoholipid chosen from lecithins, biomimetic phospholipids, and mixtures thereof.

3. The process of claim 1 wherein (a) is a polyamine chosen from a polyethyleneimine, a polyvinylamine, polyacrylate-1 crosspolymer, and mixtures thereof.

4. The process of claim 1 wherein (a) is a fatty monoamine chosen from primary, secondary, and tertiary fatty monoamines having at least one alkyl group of from about 6 to about 22 carbons, and mixtures thereof.

5. The process of claim 1 wherein (a) is chosen from stearamidopropyldimethylamine, behenamidopropyldimethylamine, and mixtures thereof.

6. The process of claim 1 wherein (a) is a fatty quaternary amine having from about 6 to about 22 carbon atoms.

7. The process of claim 1 wherein (a) is chosen from behentrimonium chloride, Quaternium-16; Quaternium-18, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dicetyldimonium chloride, behentrimonium methosulfate (18-MEA), stearalkonium chloride, and mixtures thereof.

8. The process of claim 1 wherein (a) is present in an amount of from greater than 0% to about 30% by weight, based on the total weight of the composition.

9. The process of claim 1 wherein (a) is present in an amount of from greater than 0% to about 5% by weight, based on the total weight of the composition.

10. The process of claim 1 wherein (b) has an HLB of at least about 8.

11. The process of claim 1 wherein (b) is present in an amount of from greater than 0% to about 70% by weight, based on the total weight of the composition.

12. The process of claim 1 wherein (b) is present in an amount of from greater than 0% to about 20% by weight, based on the total weight of the composition.

13. The process of claim 1 wherein (c) is an alkyl ether carboxylic acid.

14. The process of claim 1 wherein (c) is chosen from laureth-5 carboxylic acid, oleth-10 carboxylic acid, laureth-11 carboxylic acid, and mixtures thereof.

15. The process of claim 1 wherein (c) is a fatty acid having from about 6 to about 40 carbon atoms.

16. The process of claim 1 wherein (c) is chosen from oleic acid, stearic acid, and mixtures thereof.

17. The composition of claim 1 wherein (c) is chosen from PPG-5-Ceteth-10 phosphate, Oleth-3 phosphate, Oleth-10 phosphate, Ceteth-10 phosphate, Dicetyl phosphate, Cetyl phosphate, Stearyl phosphate, and mixtures thereof.

18. The process of claim 1 wherein (c) is present in an amount of from greater than 0% to about 30% by weight, based on the total weight of the composition.

19. The process of claim 1 wherein (c) is present in an amount of from greater than 0% to about 5% by weight, based on the total weight of the composition.

20. The process of claim 1 wherein (d) is chosen from silicones, natural oils, synthetic oils, hydrocarbons, polymers, and mixtures, thereof.

21. The process of claim 1 wherein (d) is present in an amount of from greater than 0% to about 50% by weight, based on the total weight of the composition.

22. The process of claim 1 wherein (d) is present in an amount of from greater than 0% to about 10% by weight, based on the total weight of the composition.

23. The process of claim 1 wherein (e) is chosen from octylacrylamide/acrylates/butylaminoethyl methacrylate copolymer, PVM/MA half ethyl ester copolymer, Vinyl acetate/crotonates/vinyl neodecanoate copolymer, t-butyl acrylate/ethyl acrylate/methacrylic acid, and acrylic acid/ethyl acrylate/t-butyl acrylamide, acrylic acid/methacrylic acid/acrylates/methacrylates, ethyl acrylate/t-butyl acrylate/methacrylic acid, acrylic acid/methacrylic acid/ acrylates/methacrylates/hydroxy ester acrylates, polyvinylpyrrolidone, vinyl acetate/vinylpyrrolidone copolymer, and mixtures thereof.

24. The process of claim 1 wherein (e) is present in an amount of from greater than 0% to about 30% by weight, based on the total weight of the composition.

25. The process of claim 1 wherein (e) is present in an amount of from greater than 1% to about 5% by weight, based on the total weight of the composition.

26. The process of claim 1 wherein the aqueous composition is present in hair styling, shampoo, conditioner, leave-in treatment, rinse-off, pre-chemical treatment and post-chemical treatment products.
